(19) United States
(12) Patent Application Publication
(10) Pub. No.: US 2017/0266157 A1
(43) Pub. Date: Sep. 21, 2017

(54) METHODS FOR WOUND HEALING AND SCAR PREVENTION

(71) Applicant: David Weinstein, Los Altos, CA (US)

(72) Inventor: David Weinstein, Los Altos, CA (US)

(21) Appl. No.: 15/453,014

(22) Filed: Mar. 8, 2017

Related U.S. Application Data
(60) Provisional application No. 62/310,909, filed on Mar. 21, 2016.
(60) Provisional application No. 62/339,388, filed on May 20, 2016.
(60) Provisional application No. 62/339,391, filed on May 20, 2016.

(51) Int. Cl. A61K 31/40; A61K 38/13; A61K 9/00

(52) U.S. Cl. CPC A61K 31/40; A61K 9/0014; A61K 38/13

(57) ABSTRACT

The present invention is related to a compound of formula (I)

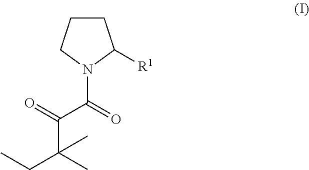

or a pharmaceutically acceptable salt or ester thereof. The present invention is further related to a method of healing and/or inhibiting formation of scar tissue in an external wound of a subject comprising topically administering to the external wound of the subject an effective amount of a compositing containing a compound that binds FK506 binding protein 4.

### FIELD OF THE INVENTION

The present invention is related to a compound of formula (I)

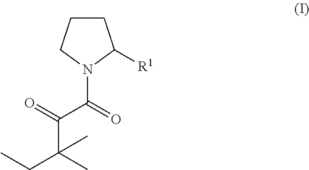

or a pharmaceutically acceptable salt or ester thereof. The present invention is further related to a method of healing and/or inhibiting formation of scar tissue in an external wound of a subject comprising topically administering to the external wound of the subject an effective amount of a compositing containing a compound that binds FK506 binding protein 4.

### BACKGROUND OF THE INVENTION

Injured mammalian tissues typically heal by a combination of regeneration and repair. Regeneration results in the re-establishment of the original tissue structure and function. In contrast, tissue repair entails the replacement of the original tissue with a patch of connective tissue, or scar, which is functionally and aesthetically inferior to the original (Ferguson M W et al., Scar-free healing: from embryonic mechanisms to adult therapeutic intervention, Philos Trans R Soc Lond B Biol Sci, 2004 May 29, 359(1445), 839-850.) The response of most mammalian tissues to injury falls within this spectrum, with some tissues that are believed not to regenerate at all. Regardless of the final outcome-scar or regenerated tissue, wound healing occurs in several stages. These stages include clot formation and release by aggregated platelets, acute inflammation, granulation and finally tissue remodeling. During coagulation, platelets release their stored granules, a rich source of growth factors and cytokines. During inflammation, white blood cells attack invading microorganisms, release additional growth factors that stimulate fibroblasts and endothelial cells, and stimulate the regeneration of superficial nerves, which are absolutely required for normal healing. During granulation, epithelial cells, fibroblasts and endothelial cells migrate to the wound site and form a cover, closing the wound. Finally during tissue remodeling, there is a competition between scar formation by fibroblasts that produce disorganized collagen and elastin fibers that contribute to scar tissue, and normal skin regeneration, in which the skin that is regenerated has similar form and function as the pre-traumatized tissue. Numerous studies have shown that skin stem cells contribute to the regeneration of damaged skin and hair (Ito M et al., Hair follicle stem cells in the lower bulge form the secondary germ, a biochemically distinct but functionally equivalent progenitor cell population, at the termination of catagen, Differentiation, 2004 December, 72(9-10), 548-557; Morris R J et al., Capturing and profiling adult hair follicle stem cells, Nat Biotechnol, 2004 April, 22(4), 411-417; Yu H et al., Isolation of a novel population of multipotent adult stem cells from human hair follicles, Am J Pathol, 2006 June, 168(6), 1879-1888.)

Regenerated normal skin has several advantages over scar tissue. In addition to cosmetic issues, scar tissue is functionally inferior to normal skin in numerous ways, including it is more susceptible to UV radiation and it is largely devoid of intradermal structures, including apocrine and sebaceous glands and hair follicles.

Cyclosporine A (“CSA”) was originally developed as an immunosuppressant to prevent solid organ graft rejection in organ transplant recipients. The immunomodulatory activity of cyclosporine is exerted through its tight binding to the calmodulin-dependent, serine/threonine protein phosphatase, calcineurin (Liu J et al., Calcineurin is a common target of cyclophilin-cyclosporin A and FKBP-FK506 complexes, Cell, 1991 Aug. 23, 66(4), 807-815.) In T cells, calcineurin/cyclosporine complexes bind to and prevent the nuclear translocation of the nuclear factor of activated T cell (“NFAT”) protein, and thus its binding to the interleukin 2 (“IL-2”) promoter. This results in a failure to activate IL-2 production, putting an early block on the cellular immune cascade (Harding, 1989; Liu et al., 1991; Schreiber S. et al., Cytokine pattern of Langerhans cells isolated from murine epidermal cell cultures, J Immunol. 1992 Dec. 1, 149(11), 3524-3534; Siekierka J J et al., FK-506 and cyclosporin A: immunosuppressive mechanism of action and beyond, Curr Opin Immunol, 1992 October, 4(5), 548-552.) A notable side effect of CSA treatment of organ allograft recipients is hair growth: 21% to 45% of solid organ allograft recipients treated with CSA developed hirsutism (Sandimmune® package insert; Sandimmune is a registered trademark of and available from Novartis AG Corp.). In addition to T cells, NFAT is also highly expressed in stem cells in a number of tissues from embryogenesis through adulthood (Friday B B et al., Calcineurin activity is required for the initiation of skeletal muscle differentiation, J Cell Biol, 2000 May 1, 149(3), 657-666; Horsley V et al., Regulation of the growth of multinucleated muscle cells by an NFATC2-dependent pathway, J Cell Biol, 2001 Apr. 16, 153(2), 329-38; Horsley V et al., NFAT: ubiquitous regulator of cell differentiation and adaptation, J Cell Biol, 2002 Mar. 4, 156(5), 771-774; Li X et al., Calcineurin-NFAT signaling critically regulates early lineage specification in mouse embryonic stem cells and embryos, Cell Stem Cell, 2011 Jan. 7, 8(1), 46-58; Zhu L et al., Foxd3 suppresses NFAT-mediated differentiation to maintain self-renewal of embryonic stem cells, EMBO Rep, 2014 December, 15(12), 1286-1296), where it suppresses stem cell differentiation (Horsley V et al., 2002.) In the skin, the stem cells that contribute to the formation of virtually all structures in the skin, including the dermis, epidermis, apocrine glands, sebaceous glands, hair follicles and hair, reside in a structure within the hair follicle termed the “bulge” (Taylor G et al., Involvement of follicular stem cells in forming not only the follicle but also the epidermis, Cell, 2000 Aug. 18, 102(4), 451-461; Yu et al. 2006.) In these cells, NFAT normally suppresses transcription of CDK4, resulting in stem cell quiescence. CSA treatment suppresses NFAT, which in turn relieves the differentiation repression on stem cells, resulting in precocious hair differentiation and anagen (hair growth) (Horsley V et al., NFATc1 balances quiescence and proliferation of skin stem cells, Cell, 2008 Jan. 25, 132(2), 299-310.) In addition to enhancing stem-cell mediated hair growth, chronic, prolonged exposure to CSA can result in the induction of pilosebaceous and mucocutaneous hypertrophy, as well as other skin hypertrophic lesions (Ponticelli C et al., Nonneoplastic mucocutaneous lesions in organ transplant recipients, Transpl Int, 2011 November, 24(11), 1041-1050; Segoloni G P et al., Renal transplantation in patients over 65 years of age: no more a contraindication but a growing indication, Transplant Proc, 2005 March, 37(2), 721-725; Menni S et al., Cutaneous and oral lesions in 32 children after renal transplantation, Pediatr Dermatol, 1991 September, 8(3), 194-198), suggesting that the effects on follicular skin cells can extend beyond acute and rapid effects on hair growth.

Another compound that has been shown to promote wound healing is RT175 (AMG-474-00, GM1485, GPI 1485). RT175 (AMG-474-00, GM1485, GPI 1485) is a 241 Dalton molecule having the following chemical structure

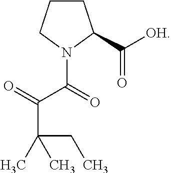 [SMILES: CCC(C)(C)C(=O)C(=O)N1CCC[C@H]1C(=O)O]

RT175 binds with high affinity to FK506 binding protein 4 (“FKBP52”). FKBP52 is known to act as a molecular chaperone for the glucocorticoid receptor (“GR”). After binding to a ligand, the RT175/GR complex translocates to the nucleus (Banerjee A., et al. Control of glucocorticoid and progesterone receptor subcellular localization by the ligand-binding domain is mediated by distinct interactions with tetratricopeptide repeat proteins, Biochemistry, 2008, 47, 10471-10480.) It has been shown that that RT175 treatment of fibroblasts for 2 hours results in the translocation of FKBP52 to the nucleus, presumably with its cargo.

Current wound healing and scar prevention compositions include several inflammation factor compositions such as activated protein C (U.S. Patent Application Publication No. 2014/0219991), hsp90a (U.S. Pat. No. 8,455,443), and FAK inhibitors (U.S. Patent Application Publication No. 2013/0165463) and extracellular matrix replacement such as hyaluronic acid (U.S. Patent Application Publication No. 2015/0064129), sodium hyaluronate (U.S. Pat. No. 8,426,384), zinc gluconate, sodium hyaluronate and collagen (U.S. Pat. No. 9,125,892.)

Despite available pharmaceutical compounds compositions, there is a continued need for new compounds, methods and compositions useful for wound healing and scar formation inhibition that utilize topically safe and effective pharmaceutical agents.

### SUMMARY OF THE INVENTION

The present invention is directed to a compound of formula (I)

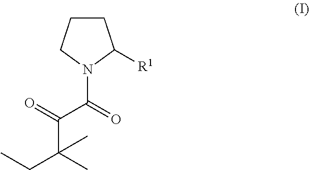

or a pharmaceutically acceptable salt or ester thereof, wherein R¹ is a methoxy, a phenyl, a benzyl, a substituted phenyl or a substituted benzyl.

In a preferred embodiment the substituted phenyl and substituted benzyl of the compound of formula (I) are each individually substituted with an alkyl group, a methoxy group or a halogen.

In a more preferred embodiment the compound of formula (I) is selected from the group consisting of

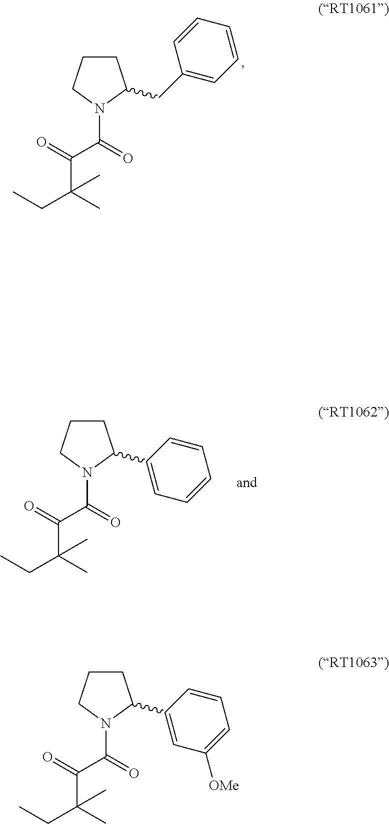 [SMILES: CCC(C)(C)C(=O)C(=O)N1CCCC1Cc1ccccc1 ; CCC(C)(C)C(=O)C(=O)N1CCCC1c1ccccc1 ; CCC(C)(C)C(=O)C(=O)N1CCCC1c1cccc(OC)c1]

and a pharmaceutically acceptable salt or ester thereof.

The present invention is further directed to topical compositions comprising a compound of formula (I) and a pharmaceutically acceptable carrier.

The present invention is further directed to a method of healing and/or inhibiting scar formation in an external wound of a subject comprising topically administering to the external wound of the subject an effective amount of a composition comprising a compound that binds FK506 binding protein 4.

In a preferred embodiment, the present invention is directed to a method of healing and/or inhibiting scar formation in an external wound of a subject comprising topically administering to the external wound of the subject an effective amount of a composition comprising a compound that binds FK506 binding protein 4 and cyclosporine A.

In another preferred embodiment, the present invention is directed to a method of healing and/or inhibiting scar formation in an external wound of a subject comprising topically administering to the external wound of the subject an effective amount of a composition comprising a compound that binds FK506 binding protein 4 and concomitantly or sequentially administering a second composition comprising cyclosporine A.

In a preferred embodiment the compound that binds FK506 binding protein 4 is a compound of formula (I)

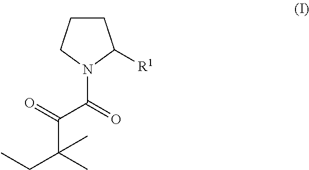

or a pharmaceutically acceptable salt or ester thereof, wherein R¹ is COOH, a methoxy, a phenyl, a benzyl, a substituted phenyl or a substituted benzyl.

In a preferred embodiment the substituted phenyl and substituted benzyl of the compound of formula (I) are each individually substituted with an alkyl group, a methoxy group or a halogen.

In a more preferred embodiment the compound of formula (I) is selected from the group consisting of

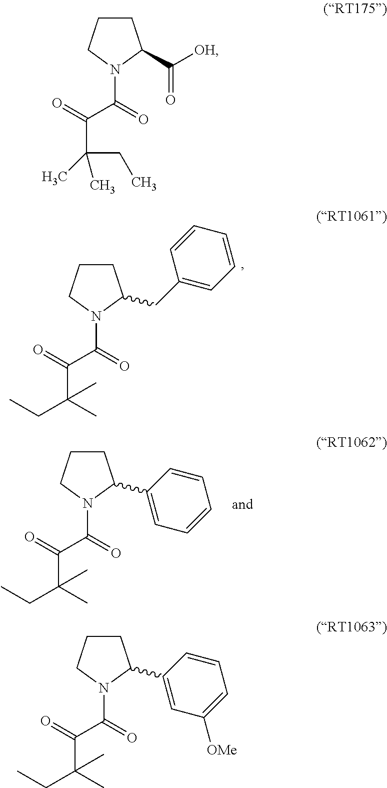

and a pharmaceutically acceptable salt or ester thereof.

In a most preferred embodiment the compound of formula (I) is RT175.

In a preferred embodiment the present invention is directed to a method of expediting the healing of an external wound of a subject comprising topically administering to the external wound of the subject an effective amount of a composition comprising a compound that binds FK506 binding protein 4.

In another preferred embodiment, the present invention is directed to a method of expediting the healing of an external wound of a subject comprising topically administering to the external wound of the subject an effective amount of a composition comprising a compound that binds FK506 binding protein 4 and cyclosporine A.

In another preferred embodiment, the present invention is directed to a method of expediting the healing of an external wound of a subject comprising topically administering to the external wound of the subject an effective amount of a composition comprising a compound that binds FK506 binding protein 4 and concomitantly or sequentially administering a second composition comprising cyclosporine A.

In another preferred embodiment the present invention is directed to a method of preventing formation of scar tissue in an external wound of a subject comprising topically administering to the external wound of the subject an effective amount of a composition comprising a compound that binds FK506 binding protein 4.

In another preferred embodiment, the present invention is directed to a method of preventing formation of scar tissue in an external wound of a subject comprising topically administering to the external wound of the subject an effective amount of a composition comprising a compound that binds FK506 binding protein 4 and cyclosporine A.

In another preferred embodiment, the present invention is directed to a method of preventing formation of scar tissue in an external wound of a subject comprising topically administering to the external wound of the subject an effective amount of a composition comprising a compound that binds FK506 binding protein 4 and concomitantly or sequentially administering a second composition comprising cyclosporine A.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Full-thickness resection of a basal cell carcinoma in a 48 year-old man treated topically with RT175.

FIG. 2. Electrodissection and curettage of basal cell sarcomas in the upper forearms of a 48 year-old man treated topically with vehicle or RT175.

FIG. 3. 6 month follow up of electrodissection and curettage of basal cell sarcomas in the upper forearms of a 48 year-old man treated topically with vehicle or RT175.

FIG. 4. 10 micron thick sections of dermal 5 mm biopsy punches in the forearm of a 62 year-old man stained for the expression of the stem cell markers Oct-4 and Sox-2.

FIG. 5. Mice mean ear-hole area following punch biopsy and 21 day treatment with RT1061.

FIG. 6. Skin regeneration and hair regrowth following shaving and rasping of mice dorsal skin and 10 day treatment with RT1061.

### DETAILED DESCRIPTION OF THE INVENTION

Applicants unexpectedly discovered a group of compounds capable of regenerating skin and regrowing hair upon topical application. Applicants further unexpectedly discovered that a composition containing a compound that binds FK506 binding protein 4 is surprisingly effective at healing an external wound and/or inhibiting the formation of scar tissue in an external wound when applied topically to the wound site. Applicant further unexpectedly discovered that adding cyclosporine A to the composition containing a compound that binds FK506 binding protein 4 also is surprisingly effective at healing an external wound and/or inhibiting the formation of scar tissue in an external wound when applied topically to the wound site.

As used herein, “RT175” refers to the compound of the formula,

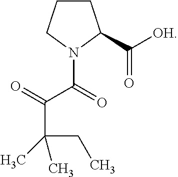 [SMILES: CCC(C)(C)C(=O)C(=O)N1CCC[C@H]1C(=O)O]

As used herein, “cyclosporine A” refers to the compound of the formula,

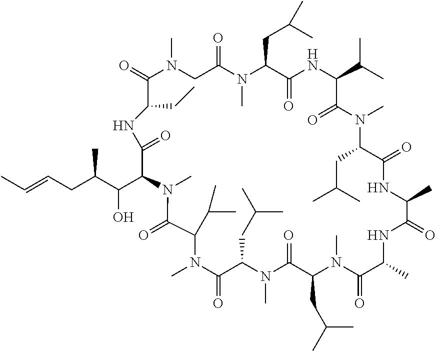 [SMILES: C/C=C/C[C@@H](C)C(O)[C@H]1C(=O)N[C@@H](CC)C(=O)N(C)CC(=O)N(C)[C@@H](CC(C)C)C(=O)N[C@@H](C(C)C)C(=O)N(C)[C@@H](CC(C)C)C(=O)N[C@@H](C)C(=O)N[C@H](C)C(=O)N(C)[C@@H](CC(C)C)C(=O)N(C)[C@@H](CC(C)C)C(=O)N(C)C(C(C)C)C(=O)N1C]

and any pharmaceutically acceptable salt or ester thereof.

As used herein the term “pharmaceutically acceptable” refers to ingredients that are not biologically or otherwise undesirable in a topical application.

As used herein the term “R¹” refers to a substituent selected from the group consisting of COOH, a methoxy, a phenyl, a benzyl, a substituted phenyl and a substituted benzyl.

In general, the term “substituted” means that one or more hydrogens of the designated moiety are replaced with a suitable substituent.

As used herein the term “alkyl” refers to a branched or straight-chain alkyl consisting of a saturated hydrocarbon group of 1 to 24 carbon atoms (C₁-C₂₄) unless otherwise stated. The alkyl group can be cyclic or acyclic.

As used herein, the term “heal” or “healing” refers to the process of the restoration of tissue to its natural state from a wounded state. Healing involves the repair of living tissue and resumption of normal functioning. During healing, cells in the body regenerate and repair to reduce the size of a damaged wound area and replace it with new living tissue. The replacement can happen in two ways: by regeneration in which damaged or missing cells are replaced by new cells that form similar tissue as was originally there; or by repair in which injured tissue is replaced with scar tissue. “Healing” may be indicated by a decrease in wound size and/or a decrease numbers of damaged cells compared to numbers of new or scar tissue cells.

As used herein, “inhibit” or “inhibiting” refers to impeding or preventing progress.

As used herein the term “expedite,” “expediting” or “expedited” refers to reducing the time of wound closure as compared to the length of time wound closure would occur without pharmaceutical intervention.

As used herein the term “external wound” refers to any breach in the epidermis and/or dermis of the subject.

The present invention is directed to a compound of formula (I)

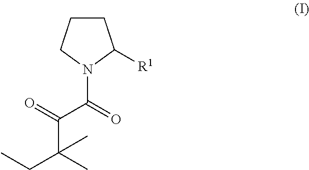

or a pharmaceutically acceptable salt or ester thereof, wherein R¹ is a methoxy, a phenyl, a benzyl, a substituted phenyl or a substituted benzyl.

In a preferred embodiment the substituted phenyl and substituted benzyl of the compound of formula (I) are each individually substituted with an alkyl group, a methoxy group or a halogen.

In a more preferred embodiment the compound of formula (I) is selected from the group consisting of

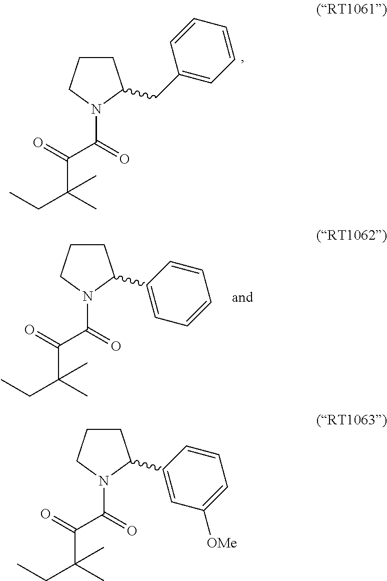

and a pharmaceutically acceptable salt or ester thereof.

The present invention is further directed to topical compositions comprising a compound of formula (I) and a pharmaceutically acceptable carrier.

The present invention is further directed to a method of healing and/or inhibiting scar formation in an external wound of a subject comprising topically administering to the external wound of the subject an effective amount of a composition comprising a compound that binds FK506 binding protein 4.

In a preferred embodiment, the present invention is directed to a method of healing and/or inhibiting scar formation in an external wound of a subject comprising topically administering to the external wound of the subject an effective amount of a composition comprising a compound that binds FK506 binding protein 4 and cyclosporine A.

In another preferred embodiment, the present invention is directed to a method of healing and/or inhibiting scar formation in an external wound of a subject comprising topically administering to the external wound of the subject an effective amount of a composition comprising a compound that binds FK506 binding protein 4 and concomitantly or sequentially administering a second composition comprising cyclosporine A.

In a preferred embodiment the compound that binds FK506 binding protein 4 is a compound of formula (I)

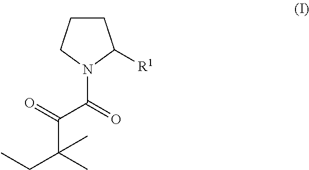

or a pharmaceutically acceptable salt or ester thereof, wherein R¹ is COOH, a methoxy, a phenyl, a benzyl, a substituted phenyl or a substituted benzyl.

In a preferred embodiment the substituted phenyl and substituted benzyl of the compound of formula (I) are each individually substituted with an alkyl group, a methoxy group or a halogen.

In a more preferred embodiment the compound of formula (I) is selected from the group consisting of

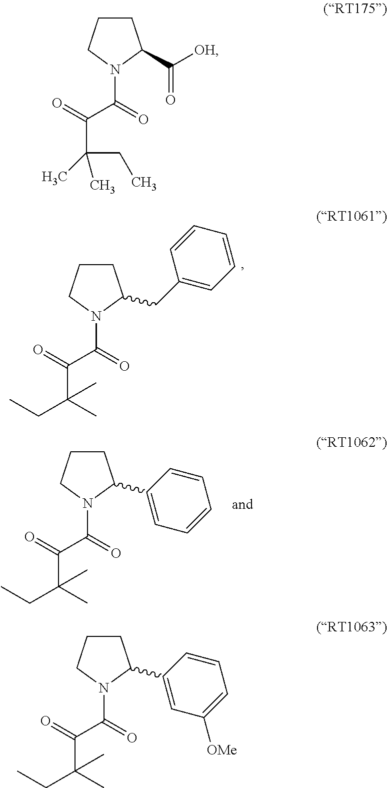

and a pharmaceutically acceptable salt or ester thereof.

In a most preferred embodiment the compound of formula (I) is RT175.

In a preferred embodiment the present invention is directed to a method of expediting the healing of an external wound of a subject comprising topically administering to the external wound of the subject an effective amount of a composition comprising a compound that binds FK506 binding protein 4.

In another preferred embodiment, the present invention is directed to a method of expediting the healing of an external wound of a subject comprising topically administering to the external wound of the subject an effective amount of a composition comprising a compound that binds FK506 binding protein 4 and cyclosporine A.

In another preferred embodiment, the present invention is directed to a method of expediting the healing of an external wound of a subject comprising topically administering to the external wound of the subject an effective amount of a composition comprising a compound that binds FK506 binding protein 4 and concomitantly or sequentially administering a second composition comprising cyclosporine A.

In another preferred embodiment the present invention is directed to a method of preventing formation of scar tissue in an external wound of a subject comprising topically administering to the external wound of the subject an effective amount of a composition comprising a compound that binds FK506 binding protein 4.

In another preferred embodiment, the present invention is directed to a method of preventing formation of scar tissue in an external wound of a subject comprising topically administering to the external wound of the subject an effective amount of a composition comprising a compound that binds FK506 binding protein 4 and cyclosporine A.

In another preferred embodiment, the present invention is directed to a method of preventing formation of scar tissue in an external wound of a subject comprising topically administering to the external wound of the subject an effective amount of a composition comprising a compound that binds FK506 binding protein 4 and concomitantly or sequentially administering a second composition comprising cyclosporine A.

In a preferred embodiment compositions of the present invention further comprise a pharmaceutically acceptable carrier, preferably the pharmaceutically acceptable carrier is Amantle™ (Amantle is available from Doak Dermatologics), more preferably the pharmaceutically acceptable carrier comprises water, ceteryl alcohol, sodium lauryl sulfate, sodium cetearyl sulfate, petrolatum, glycerin, synthetic beeswax, mineral oil, methylparaben, aluminum sulfate, calcium acetate and white potato dextrin.

The following examples are for are provided solely for illustrative purposes and are not meant to limit the invention in any way.

### Examples

### Example 1. Rapid, Scar-Free Healing Following RT175 Treatment of the Resection Site of a Periauricular Basal Cell Carcinoma

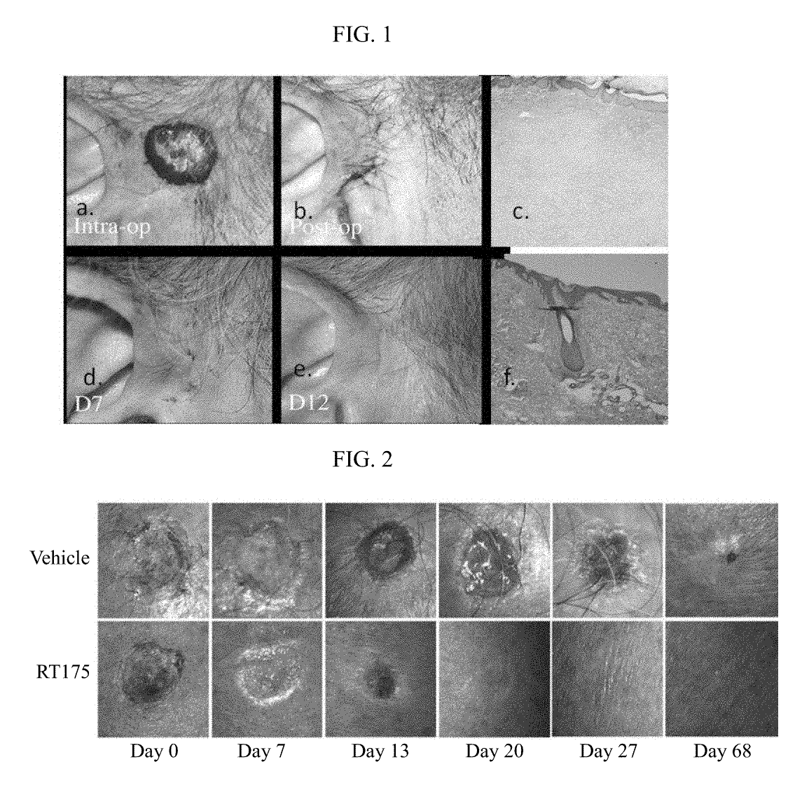

A 48 year-old man underwent a full-thickness resection of a basal cell carcinoma on an outpatient basis. See FIG. 1, panel a. showing wound during operation and panel b. showing wound immediately post operation. RT175 formulated in an Amantle™ cream base was applied immediately post-operatively, and then daily thereafter for 12 days. Surprisingly, by the 7^{th} post-operative day, the wound was closed and largely healed. See FIG. 1, panel d. By the 12^{th} post-operative day, the skin appeared to have returned to baseline, with little to no scar observed. See FIG. 1, panel e. The absence of scar tissue was confirmed by biopsy 6 months later. See FIG. 1, panel c. An archival photo of a scar biopsy is included for comparison. See FIG. 1, panel f.

### Example 2. Rapid, Scar-Free Healing Following Electrodissection and Curettage Treatment of the Upper Arm for the Treatment of Basal Cell Carcinomas

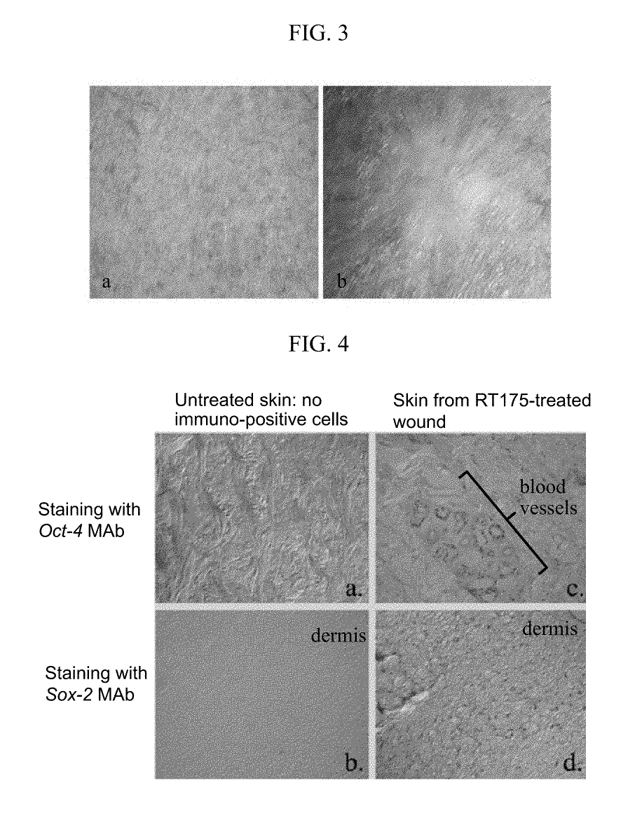

The supradeltoid regions of the left and right upper arm were treated by electrodissection and curettage (“ED&C”) for treatment of basal cell sarcomas. See FIG. 2, Day 0. Beginning on the day of surgery, and daily thereafter the sites were treated with RT175 in an Amantle™ cream base, or with the Amantle™ cream base alone. Surprisingly, by the 20^{th} post-operative day, the RT175 treated site was fully closed. See FIG. 2, Day 20, bottom panel. In contrast, 68 days after the procedure the vehicle treated site was still not fully closed. See FIG. 2, Day 68, top panel. Six-months following the procedure, the RT175-treated site was scar-free, see FIG. 3, panel a., while the vehicle-treated site has a pronounced, hypopigmented stellate shaped scar, see FIG. 3, panel b.

### Example 3. Treatment of Wounds with RT175 Induces Stem Cells at the Site of Treatment

A 62 year-old male underwent bilateral 5 millimeter (“mm”) dermal biopsy punches in the forearm, 5 centimeter (“cm”) distal to the antecubital space. Treatment with RT175 in an Amantle™ cream base, or with the Amantle™ cream base alone was begun immediately upon hemostasis. The skin was treated daily for 30 days, whereupon the sites were re-biopsied and the tissue processed for immunohistology. 10 micron thick sections were stained for the expression of the stem cell markers Oct-4, see FIG. 4, panels a. and c., or Sox-2, see FIG. 4, panels b. and d. Subsets of cells in the RT175 treated tissues, see FIG. 4, panels c. and d. expressed both stem cell markers, while no cells in the vehicle-treated tissue, see FIG. 4, panels a. and b. expressed either marker. Not to be held to a particular theory, the presence of stem cells markers in RT175 treated wound sites indicates active stem cells. The presence of these stem cells in the wound site causes rapid and proper wound healing without the formation of scar tissue. In turn, the absence of stem cells from wound sites allows the formation of scar tissue.

### Example 4. Treatment of Punch-Hole Biopsies of the Ear of Mice with RT175 Analog, RT1061

Retired ICR breeder mice of both sexes with weight between 45 and 50 grams were subject to single bilateral punch-hole biopsies. The animals were then randomly assigned to one of three groups to receive either 2.4 μg of RT1061, 80 μg FK506 or a vehicle control. RT1061, FK506 or vehicle control was applied topically to both the ventral and dorsal surface of the ear, once each day including immediately after biopsy.

All wounds were photographed using a Nikon CoolPix 990 digital camera. Each image included a metric ruler that had been approximated to the wound. The images of the wounds were opened in Adobe Photoshop, and using the “measurement tool”, the length, in pixels, of 3 cm along the ruler's straight edge was determined. That number was used to calculate a conversion factor that was then used to normalize each photograph. The conversion factor was calculated as 100/length. To normalize the area of the lesion in a photograph, the volume of the lesion was determined in voxels using the “magic wand” tool, and the number of voxels were multiplied by the conversion factor, thus generating the equation: area=volume (voxels)*conversion factor. The area at t=0 was defined as 100%. To calculate the percentage of an open area of any given ear-hole we applied the following equation: 100[area (t=x)/area (t=0)]. The mean area of the initial hole was calculated as average radii of the punches at t=0 (determined by using an outline of the hole measured against the ruler, using Adobe Photoshop) times π. As an example, a given radius of 1.28 mm gives values of π*1.28²=5.15 mm².

Results of the study can be seen in FIG. 5, which displays mean ear-hole area for all mice treated with each particular composition. Topical application of RT1061 to the wound site led to almost complete regeneration of the mouse pinna including hair follicles and glands, blood vessels, cartilage, nerves and muscles, within 21 days of the punch-hole biopsy. In contrast, topical application of FK506 or control vehicle did not lead to regeneration of mouse pinna tissue.

### Example 5. Treatment of Dermabrasion Lesions of Dorsal Skin of Mice with RT175 Analog, RT1061

Retired ICR breeder mice of both sexes with weight between 45 and 50 grams were shaved and rasped to induce dermabrasion lesions. The animals were then randomly assigned (5 per treatment arm) to one of two groups to receive either 5 μL of 100 nanomolar RT1061 or a vehicle control. RT1061 or vehicle control was applied topically to the lesion site once each day including immediately after dermabrasion.

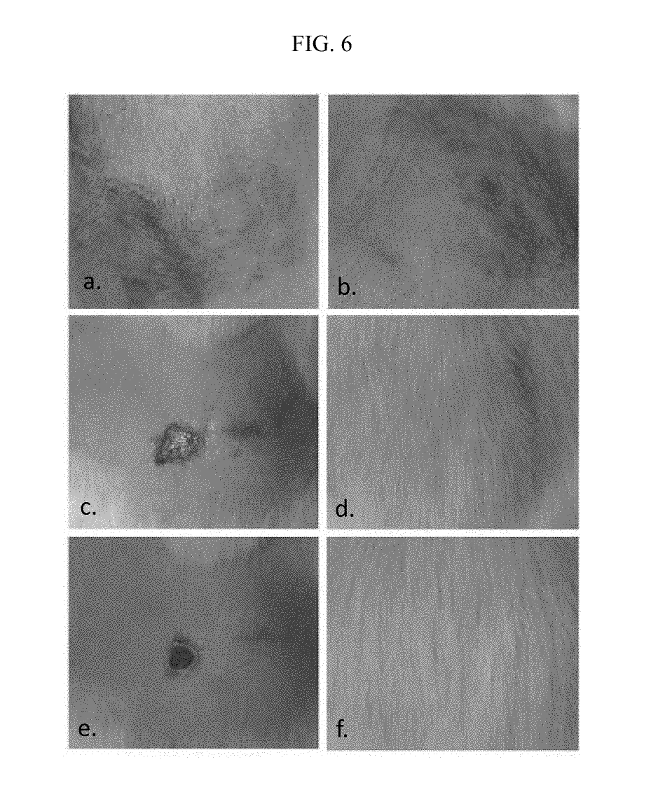

Results of the study can be seen in FIG. 6. As shown, treatment with RT1061 (panels b., d. and f.) resulted in rapid hair growth and rapid regeneration of rasped skin. In contrast, treatment with the control vehicle resulted in scab formation and no hair regrowth 10 days after rasping. See FIG. 6, panels a., c. and e.

### Example 6. Synthesis of RT1061

The following methods are used for the synthesis of either the (S)-isomer, (R)-isomer or racemic (i.e., (R,S)-isomers) compounds shown in Scheme I depending on the chirality of the starting materials. If (S)-prolinol was used to synthesize compound 4 then all resulting compounds were the (S)-isomer. If (R)-prolinol was used to synthesize compound 4 then all resulting compounds were the (R)-isomer. If both (S) and (R)-prolinol were used to synthesize compound 4 then all resulting compounds were racemic.

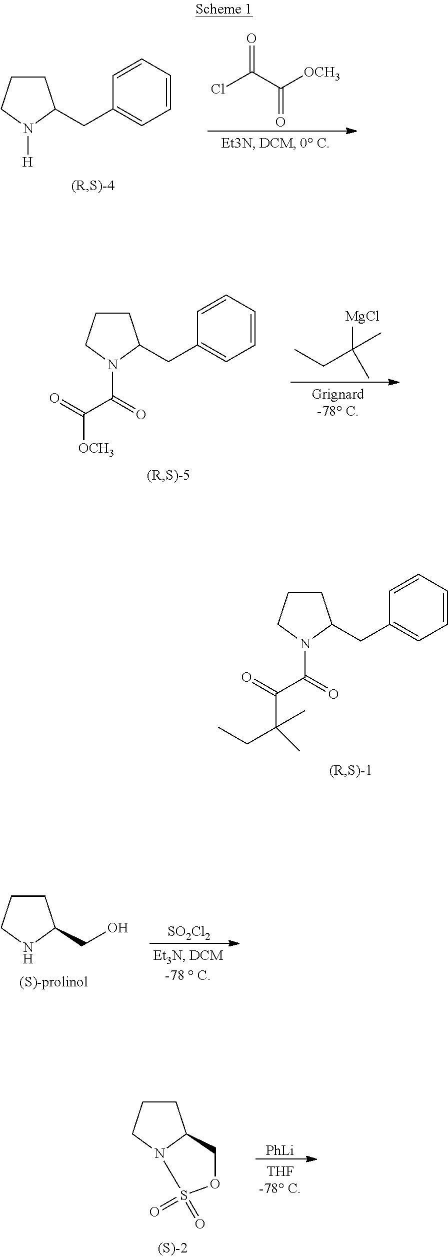

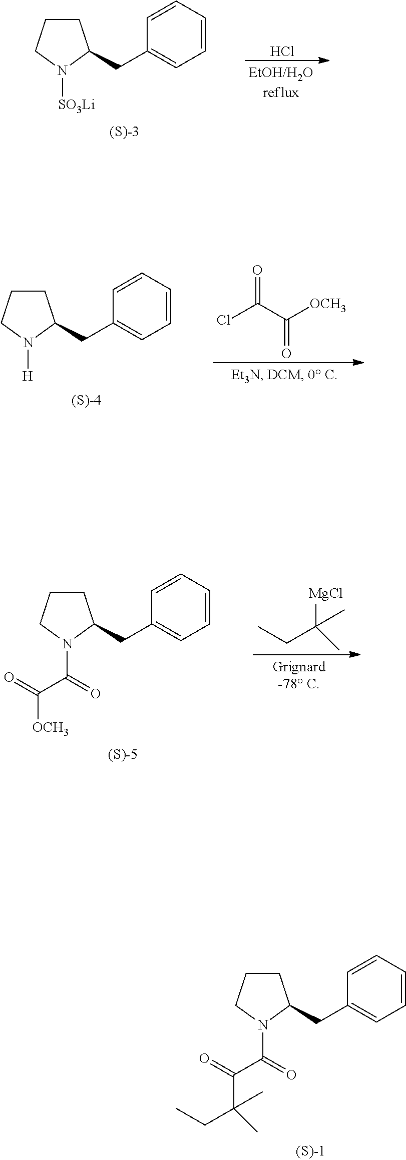

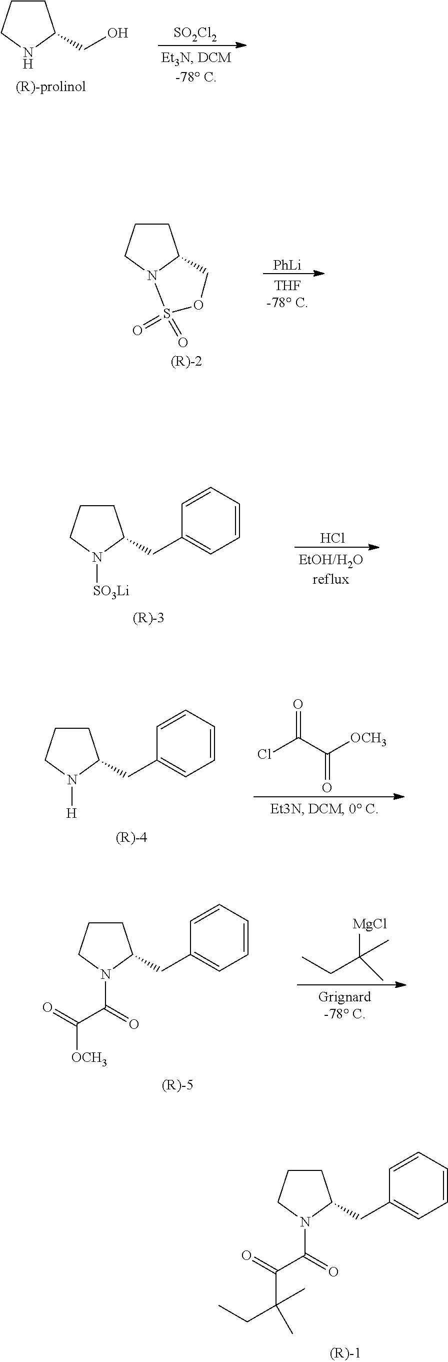

### Synthesis of (S)-2-Benzyl pyrrolidine (compound (S)-4) from (S)-prolinol

A preparation of sulphamidate (method of Alker, D.; Doyle, K. J.; Harwood, L. M.; McGregor, A.; Tetrahedron Asymmetry 1990, 1, 877.) (S)-prolinol (Aldrich) (2.62 g, 25.2 mmol) and triethylamine (“Et3N”; 7.0 mL, 50.0 mmol) were dissolved in 150 mL dry dichloromethane (“DCM”) under argon in a 500 mL round bottom flask and cooled to −78° C. To this mixture was added sulfuryl chloride (“SO₂Cl₂”; 2.1 mL, 25.9 mmol) in 150 mL dry dichloromethane dropwise over 45 minutes and stirred at −78° C. for 3 hours then allowed to warm to room temperature and stirred overnight. The mixture was washed 0.1 N HCl (3×75 mL), brine (75 mL), dried over sodium sulfate, filtered and the solvent removed via vacuum resulting in the sulfamidate (Compound (S)-2) a yellow oil (2.66 g).

The crude product was purified by applying to a dry silica cartridge (Analogix, 40 g) in 7 mL dichloromethane and eluting with a stepwise gradient of ethyl acetate in hexanes (0, 10, 20%). The fractions containing product were combined and concentrated to dryness. Upon further drying colorless crystals formed to yield 1.36 g (33%) of compound (S)-2. The product was characterized by 1H NMR and ms (APCI) 164 (M+H)+.

Bromobenzene (0.265 mL, 2.53 mmol) was dissolved in 2 mL dry THF in a 10 mL round bottom flask under argon and cooled to −78° C. n-Butyllithium (0.96 mL of 2.5 M solution in hexanes, 2.40 mmol) was added via syringe over 40 minutes and the reaction was stirred for 1 h at −78° C. 0.326 g (2.00 mmol) of 2 in 1.5 mL THF was added to this mixture via syringe over 10 minutes and the reaction was allowed to warm to room temperature and stirred overnight. The reaction was quenched with a few drops of water and the solvent removed via vacuum to yield a tan viscous oil (compound 3). This residue was dissolved in 10 mL of a 1:1 solution of 2 N HCl/ethanol and heated at reflux for 22 hours. After cooling the ethanol was removed via vaccum, 5 mL 0.1 N HCl added and the aqueous solution was extracted with ethyl acetate (3×5 mL). The pH of the aqueous solution was adjusted to >10 with 50% sodium hydroxide (“NaOH”) and extracted with ethyl acetate (4×5 mL). The ethyl acetate layers were combined and washed with brine, dried and the solvent removed via vacuum resulting in 0.262 g of compound (S)-4 a light brown oil (81%). The product was characterized by 1H NMR and ms (ESI): 162 (M+H)+.

### Synthesis of (R)-2-Benzyl pyrrolidine (compound (R)-4) from (R)-prolinol

The same procedures were followed as in the synthesis of (S)-2-Benzyl pyrrolidine (compound (S)-4) from (S)-prolinol except (R)-prolinol was substituted for (S)-prolinol.

### Synthesis of Methyl 2-(2-benzylpyrrolidine-1-yl)-2-oxoacetate (compound 5)

52 milligrams (“mg”; 0.324 millimoles (“mmol”)) of 2-benzylpyrrolidine (compound 4; ASDI Inc.) was dissolved in 2 milliliters (“mL”) dry dichloromethane in a 2 dram vial and cooled to 0° C. Triethylamine (68 μL, 0.488 mmol) and methyl chlorooxoacetate (47 μL, 0.485 mmol) were added and the reaction stirred at 0° C. for 1 hour and then room temperature overnight. The reaction mixture was diluted with 5 mL dichloromethane, washed with 0.1 N HCl (3×3 mL), saturated sodium bicarbonate solution (3×3 mL), dried and the solvent removed via vacuum to yield 61 mg of compound 5 as a tan viscous oil (76%). The product was characterized by 1H nuclear magnetic resonance (“NMR”) spectroscopy and liquid chromatography-mass spectrometry (“lc/ms”; ESI): 248 (M+H)+ and used directly for the next reaction.

### Synthesis of 1-(2-benzylpyrrolidin-1-yl)-3,3-dimethylpentane-1,2-dione (compound 1; RT1061)

61 mg (0.247 mmol) of compound 5 was dissolved in 2 mL dry tetrahydrofuran (“THF”) under argon in a 2 dram vial fitted with a Teflon® septa cap and cooled to −78° C. 0.37 mL of 1,1-dimethylpropane magnesium chloride (1 M ether solution, Aldrich) was added via syringe and the reaction stirred at −78° C. for 2 hours and then allowed to warm to room temperature and stirred overnight. The reaction was quenched by the addition of aqueous ammonium chloride and the aqueous mixture was extracted with ethyl acetate (3×5 mL). The ethyl acetate was washed with brine (1×), dried and the solvent removed via vacuum to yield a light tan oil. The product (compound 1) was purified by applying an ethyl acetate solution to a dry silica cartridge (Analogix, 4 g) and eluting with hexanes and then 2% ethyl acetate/hexanes. 35 mg of compound 1 as a colorless viscous oil was isolated and characterized by 1H NMR and lc/ms (ESI): 288.1 (M+H)+.

### Entantiomeric Purity

The enantiomeric purity of the compounds at the key stage of the 2-benzylpyrrolidine isomers (compound 4) was estimated using Mosher's reagent to produce the diastereomeric Mosher amides, which were separable by high pass liquid chromatography and indicated that the synthesis of the chiral compounds 4 proceeded to give compounds of >95% isomeric purity. (method of J. A. Dale, D. L. Dull, H. S. Mosher (1969). “α-Methoxy-α-trifluoromethylphenylacetic acid, a versatile reagent for the determination of enantiomeric composition of alcohols and amines”. Journal of Organic Chemistry 34 (9): 2543-2549.

What is claimed is:

1. A method of healing an external wound of a subject comprising topically administering to the external wound of the subject an effective amount of a composition comprising a compound that binds FK506 binding protein 4.

2. The method of claim 1 wherein the composition further comprises cyclosporine A.

3. The method of claim 1, wherein the compound that binds FK506 binding protein 4 is a compound of formula (I) 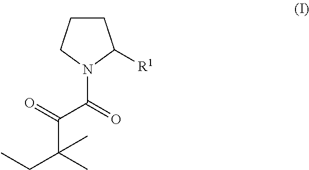
or a pharmaceutically acceptable salt or ester thereof,
wherein R¹ is selected from the group consisting of COOH, a methoxy, a phenyl, a benzyl, a substituted phenyl and a substituted benzyl.

4. The composition of claim 3 wherein the substituted phenyl and the substituted benzyl are each individually substituted with an alkyl group, a methoxy group or a halogen.

5. The composition of claim 3 wherein the compound of formula (I) is selected from the group consisting of 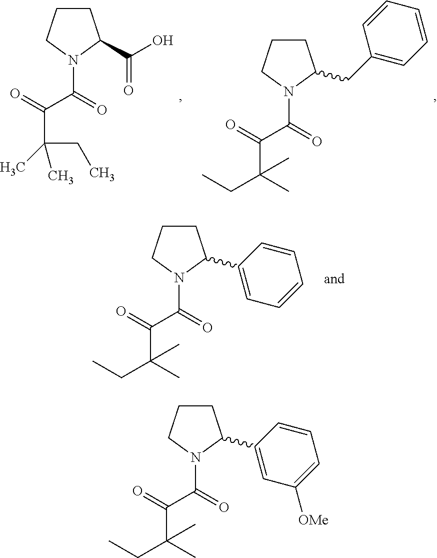
and a pharmaceutically acceptable salt or ester thereof.

6. The composition of claim 5, wherein the compound of formula (I) is a compound of formula (II)

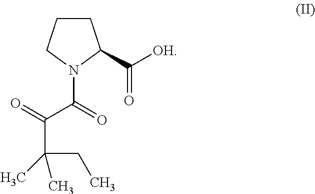 [SMILES: CCC(C)(C)C(=O)C(=O)N1CCC[C@H]1C(=O)O]

7. The method of claim 1, wherein the healing of the external wound is expedited.

8. The method of claim 1, wherein the composition further comprises a pharmaceutically acceptable carrier.

9. The method of claim 1, further comprising the concomitant or sequential administration of a second composition comprising cyclosporine A.

10. The method of claim 1, wherein formation of scar tissue is inhibited.

11. The method of claim 10 wherein formation of scar tissue is prevented.

12. A compound of formula (I) 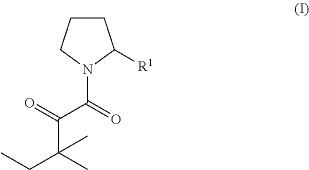
or a pharmaceutically acceptable salt or ester thereof, wherein R¹ is a methoxy, a phenyl, a benzyl, a substituted phenyl or a substituted benzyl.

13. The compound of claim 12, wherein the substituted phenyl and substituted benzyl of the are each individually substituted with an alkyl group, a methoxy group or a halogen.

14. The compound of claim 12, wherein the compound is

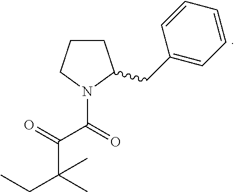 [SMILES: CCC(C)(C)C(=O)C(=O)N1CCCC1Cc1ccccc1]

15. The compound of claim 12, wherein the compound is

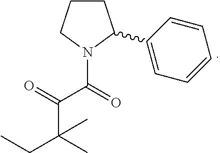 [SMILES: CCC(C)(C)C(=O)C(=O)N1CCCC1c1ccccc1]

16. The compound of claim 12, wherein the compound is

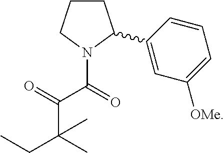 [SMILES: CCC(C)(C)C(=O)C(=O)N1CCCC1c1cccc(OC)c1]

17. A topical composition comprising a compound of claim 12 and a pharmaceutically acceptable carrier.
